(19) United States
(12) Patent Application Publication
(10) Pub. No.: US 2021/0308344 A1
(43) Pub. Date: Oct. 7, 2021

(54) Blood Bag

(71) Applicant: AI Biomaterial Healthtech Ltd., Kwun Tong KLN (HK)

(72) Inventor: Yen-Yi Ho, Taoyuan City (TW)

(21) Appl. No.: 17/095,951

(22) Filed: Nov. 12, 2020

(30) Foreign Application Priority Data
Nov. 8, 2019 (TW) 108140667

(51) Int. Cl. A61M 1/02; A61M 1/38

(52) U.S. Cl. CPC A61M 1/0259; A61M 1/385; A61M 2205/3368; A61M 2205/02; A61M 2205/3553; A61M 2205/8206; A61M 2205/60

(57) ABSTRACT

A blood bag for containing a blood and electrically connected to a blockchain network is disclosed. The blood bag includes a bag, a chip set and a blood property sensing module. The bag contains the blood. The chip set in located on the bag. The chip set includes a blockchain blood tracer for recording the source of the blood and a transportation history in the blockchain network for tracing. The blood sensing module is located on the bag and electrically connected to the chip set. The blood property sensing module is used for sensing at least one blood property of the blood.

### BACKGROUND OF THE INVENTION

### 1. Field of the Invention

The present invention relates to a blood bag; more particularly, the present invention relates to a blood bag that can trace the source of blood.

### 2. Description of the Related Art

When ordinary people donate blood, the blood drawn will be stored in a blood bag and used to help others. However, some blood donors may have blood borne diseases (such as AIDS, hepatitis, or malaria). It is not possible to confirm whether the blood has the virus at the moment of blood donation, and is necessary to conduct blood test after donation to confirm whether the blood has virus.

However, the existing blood donation and preservation system can only confirm whether the existing blood has the virus, and it is difficult to trace the identity of the donor from the blood, so it is hard to notify the donor that he or she gets sick. Therefore, how to provide a blood storage device that can trace the source of blood has become a topic worthy of exploration.

### SUMMARY OF THE INVENTION

The main purpose of the present invention is to provide a blood bag that can trace the source of blood.

In order to achieve the above-mentioned purpose, a blood bag of the present invention is used for containing a blood and electrically connected with a blockchain network. The blood bag includes a bag, a chip set and a blood property sensing module. The bag contains the blood. The chip set is arranged on the bag, and includes a blockchain blood tracer, which is used for recording the source of the blood and the transportation history of the blood bag on the blockchain network for tracing. The blood property sensing module is arranged on the bag and electrically connected to the chip set, and used for sensing at least one blood property of the blood.

According to an embodiment of the present invention, the chipset further includes a positioning element for positioning the blood bag.

According to an embodiment of the present invention, the chipset further includes a temperature detecting element for detecting the temperature of the blood bag.

According to an embodiment of the present invention, the chipset further includes an electronic tag element, which records the information of the blood bag by means of radio frequency identification.

According to an embodiment of the present invention, the blood bag further includes a wireless module, which is arranged on the bag and electrically connected to the chip set, the blood property sensing module and an external computer.

According to an embodiment of the present invention, the blood bag further includes a battery, which is arranged on the bag and provides power to the chip set, the blood property sensing module and the wireless module.

According to an embodiment of the present invention, the bag is made of biodegradable plastic.

According to an embodiment of the present invention, the positioning element is a global positioning system chip.

According to an embodiment of the present invention, the source of blood includes the identity of the blood donor, and the transportation history includes the location of the blood donation, the blood processing institution and the process of storing and transporting the blood bag.

### BRIEF DESCRIPTION OF THE DRAWINGS

FIG. 1 is a schematic diagram of the blood bag in an embodiment of the present invention.

FIG. 2 is a system architecture diagram of the blood bag and an external computer in an embodiment of the present invention.

### DETAILED DESCRIPTION OF THE PREFERRED EMBODIMENT

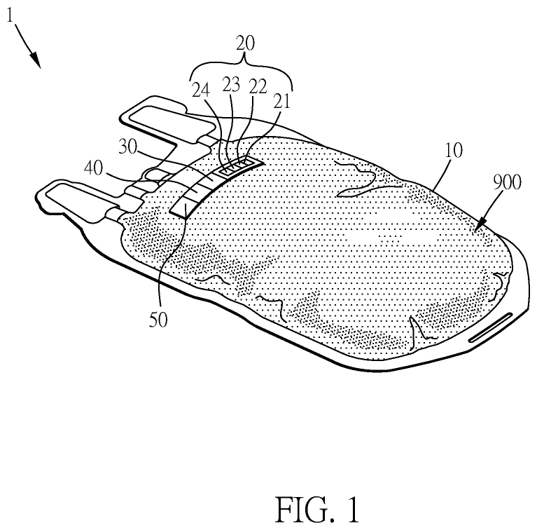

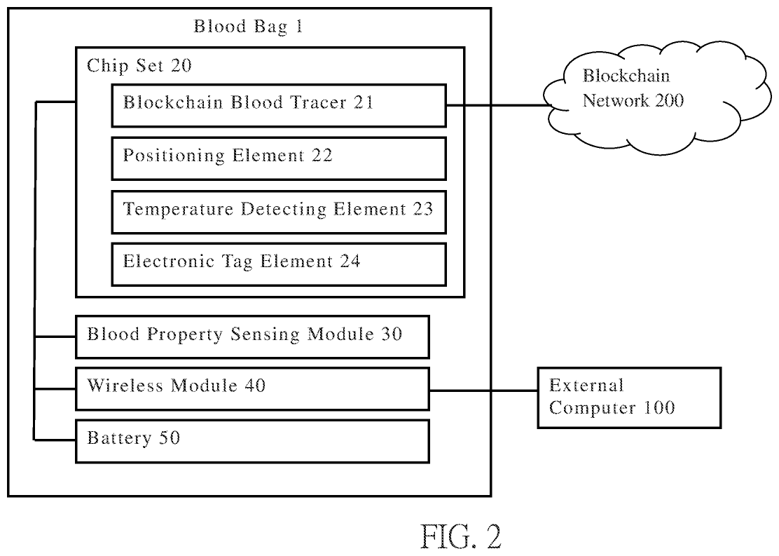

Referring to FIG. 1 and FIG. 2 for the blood bag of an embodiment of the present invention, FIG. 1 is a schematic diagram of the blood bag in an embodiment of the present invention; FIG. 2 is a system architecture diagram of the blood bag and an external computer in an embodiment of the present invention.

As shown in FIG. 1 and FIG. 2, in an embodiment of the present invention, the blood bag 1 is used for containing the blood 900, and the blood bag 1 can trace the source and history of the blood 900. The blood bag 1 can be electrically connected to an external computer 100 to transmit the relevant information of the blood bag 1 and the blood 900 contained therein to the external computer 100. The blood bag 1 includes a bag 10, a chip set 20, and a blood property sensing module 30.

In an embodiment of the present invention, the bag 10 is used for containing the blood 900. The bag 10 is made of biodegradable plastic. Biodegradable plastic is a plastic composed of microbial decomposable materials such as starch, polylactic acid, and fiber protein, so the bag 10 of the present invention can be decomposed by microorganisms and has an effect of environmental protection.

In an embodiment of the present invention, the chip set 20 is arranged on the bag 10. The chip set 20 includes a blockchain blood tracer 21, a positioning element 22, a temperature detecting element 23, and an electronic tag element 24. The blockchain blood tracer 21 is a computing chip, which is electrically connected with a blockchain network 200. The blockchain blood tracer 21 records the source and history of the blood 900 on the blockchain network 200. The transportation history of blood bag 1 is recorded and traced by the blockchain which has the characteristics of tracing and not easily being tampered with. According to one embodiment of the present invention, the source of the blood 900 includes the identity of the donor of the blood 900, and the transportation history includes the location of the blood donation, the institution that processes the blood 900 in the blood bag 1, and the process of storing and transporting the blood bag 1. In addition, the transportation history of blood bag 1 can also be entered into the blockchain blood tracer 21 by the external computer 100, and then recorded on the blockchain network 200 by the blockchain blood tracer 21. In this way, the source of blood 900 in blood bag 1 and the transportation history after the donation can be clearly recorded and managed by the blockchain which has the characteristics of tracing and not easily being tampered with. The positioning element 22 is a global positioning system chip, and used to position the blood bag 1 to determine the current position of the blood bag 1. The temperature detecting element 23 is a chip with a temperature sensing function for detecting the temperature of the blood bag 1 and monitoring whether the blood bag 1 is at an appropriate temperature to prevent overheating from affecting the quality of the blood 900. The electronic tag element 24 records the information of the blood bag 1 by means of radio frequency identification, such as the information of the capacity of the blood bag 1, the expiration date of the bag 10, the identity of the blood donor, the date and location of the blood donation, and the blood type of the blood 900. The information recorded by the electronic tag element 24 can be input by the external computer 100. Nevertheless, the information of blood bag 1 is not limited to the above, and it can be altered according to the design requirements.

In an embodiment of the present invention, the blood property sensing module 30 is arranged on the bag 10 and electrically connected to the chip set 20. The blood property sensing module 30 is a chip with a blood property measurement function for measuring at least one blood property of blood 900, such as blood viscosity, pH, blood specific gravity, plasma permeability, and blood osmotic pressure, etc. With the efficacy of measuring the blood properties by the blood property sensing module 30, that the blood 900 is in a good or deteriorated condition can be confirmed. However, the blood properties measured by the blood property sensing module 30 are not limited to the above, and it can be altered according to the design requirements.

In an embodiment of the present invention, the wireless module 40 is, for example, a network chip. The wireless module 40 is arranged on the bag 10 and electrically connected to the chip set 20, the blood property sensing module 30, and the external computer 100. The wireless module 40 is used for transmitting the information measured by the chip set 20 and the blood property sensing module 30 to the external computer 100, such that the medical staff can operate the external computer 100 to understand the data of the blood 900 contained in the blood bag 1, such as the source, history and nature of the blood.

In an embodiment of the present invention, the battery 50 is arranged in the bag 10 and provides power to the chip set 20, the blood property sensing module 30, and the wireless module 40 to ensure that the power is supplied to the chip set 20, the blood property sensing module 30, and the wireless module 40 for normal operation.

As shown in FIG. 1 and FIG. 2, when the medical staff uses the blood bag 1 of the present invention to store blood 900 donated by a blood donor, the drawn blood 900 will be placed in the bag 10, and the blood property sensing module 30 will measure the blood properties of the blood 900 in the bag 10, such as blood viscosity, pH, blood specific gravity, plasma permeability, and blood osmotic pressure. In this way, that the blood 900 is in a good or deteriorated condition can be confirmed.

In addition, the positioning element 22 is used for positioning the blood bag 1 to determine the current position of the blood bag 1 and preventing the blood bag 1 from being lost. The temperature detecting element 23 is a chip with a temperature sensing function for detecting the temperature of the blood bag 1 and monitoring whether the blood bag 1 is at a suitable temperature to prevent overheating from affecting the quality of the blood 900.

In addition, the medical staff can also connect the external computer 100 to the wireless module 40 of the blood bag 1 for inputting the information of the blood bag 1 to the electronic tag element 24, such as the information of the capacity of the blood bag 1, the expiration date of the bag 10, the identity of the blood donor, the date and location of the blood donation, and the blood type of the blood 900, etc. In this way, when the blood 900 in the blood bag 1 is used for blood transfusion in the future, the medical staff can use the radio frequency identification reader to read the data recorded by the electronic tag element 24 to quickly understand the relevant information of blood bag 1 and blood 900.

In addition, the medical staff can also operate the external computer 100 to input the article transportation history information into the blockchain blood tracer 21, and the blockchain blood tracer 21 then records the article transportation history information to the blockchain network 200. Through the function of the blockchain to trace the transportation history of the article, information such as the identity of the blood donor corresponding to blood bag 1, the place of blood donation, the institution handling blood 900 in the blood bag 1, and the process of storing and transporting blood bag 1, etc. can be recorded in the blockchain network 200. In this way, it is possible to clearly record and manage the source of blood 900 in blood bag 1 and the transportation history after donation by the blockchain which has the characteristics of tracing and not easily being tampered with. Thus, even if the blood 900 is detected to have virus, the medical staff can quickly trace the identity of the blood donor through the blockchain to trace the identity of the donor. In addition, if the blood donor has an accident and requires surgery after the blood donation, the medical staff can also trace the transportation history of blood bag 1 through the blockchain and quickly find the blood bag containing the blood 900 donated by that blood donor 1, and use the donor's own blood 900 for surgery, thereby reducing the probability of blood transfusion rejection.

With the design of the blood bag 1 of the present invention, the blood bag 1 has diversified and convenient functions, such as tracing the source and history of blood, positioning the blood bag 1 to determine the current position of the blood bag 1, detecting the temperature of the blood bag 1 to monitor whether the blood bag 1 is at the appropriate temperature, recording the blood data with the radio frequency identification method so that the medical staff can quickly read the data, and measuring the blood properties of blood. In addition, the bag is made of biodegradable plastic, which can be decomposed by microorganisms in the natural environment, so it has an effect of environmental protection.

Although the invention has been explained in relation to its preferred embodiment, it is to be understood that many other possible modifications and variations can be made without departing from the spirit and scope of the invention as hereinafter claimed.

What is claimed is:

1. A blood bag, used for containing a blood and electrically connected with a blockchain network, the blood bag comprising:
a bag, used for containing the blood;
a chip set, arranged on the bag and comprising a blockchain blood tracer, wherein the blockchain blood tracer is used for recording a source of the blood and a transportation history of the blood bag on the blockchain network for tracing; and
a blood property sensing module, arranged on the bag and electrically connected to the chip set, for sensing at least one blood property of the blood.

2. The blood bag defined in claim 1, wherein the chipset further comprises a positioning element for positioning the blood bag.

3. The blood bag defined in claim 2, wherein the chipset further comprises a temperature detecting element for detecting a temperature of the blood bag.

4. The blood bag defined in claim 3, wherein the chipset further comprises an electronic tag element, which records an information of the blood bag by means of a radio frequency identification.

5. The blood bag defined in claim 4, wherein the blood bag further comprises a wireless module, which is arranged on the bag body and electrically connected to the chip set, the blood property sensing module and an external computer.

6. The blood bag defined in claim 5, wherein the blood bag further comprises a battery, which is arranged on the bag and provides power to the chip set, the blood property sensing module, and the wireless module.

7. The blood bag defined in claim 6, wherein the bag is made of biodegradable plastic.

8. The blood bag defined in claim 7, wherein the positioning element is a global positioning system chip.

9. The blood bag defined in claim 1, wherein the source of the blood comprises an identity of a blood donor, and the transportation history comprises a location of a blood donation, a blood processing institution and a process of storing and transporting the blood bag.
